(19) United States
(12) Patent Application Publication
(10) Pub. No.: US 2021/0307759 A1
(43) Pub. Date: Oct. 7, 2021

(54) DOUBLE BALLOON FLUID-CARRYING CATHETER

(71) Applicant: Yihan CHI, Ruian (CN)

(72) Inventor: Yihan CHI, Ruian (CN)

(21) Appl. No.: 17/221,582

(22) Filed: Apr. 2, 2021

(30) Foreign Application Priority Data
Apr. 2, 2020 (CN) 20201024704.7

(51) Int. Cl. A61B 17/12

(52) U.S. Cl. CPC A61B 17/12045; A61M 2025/1097; A61B 17/12136; A61B 17/12109

(57) ABSTRACT

Disclosed is a double balloon fluid-carrying catheter including an outer conduit and a balloon. Two Luer ports are provided at a proximal end of the outer conduit, of which one is used to inflate and deflate the balloon and the other is used to flush a guidewire lumen, and the balloon is provided near a distal end of the outer conduit, wherein the balloon is provided with two balloons, namely a proximal balloon and a distal balloon, and there is a certain distance between the proximal balloon and the distal balloon; a proximal end of the proximal balloon is provided with a plurality of side holes on a wall of the outer conduit, the side holes conduct an inside and an outside of the outer conduit, and a distal end of the distal balloon is provided with a bridging reflow hole.

### TECHNICAL FIELDS

The present invention relates to a medical device, in particular, to a double balloon fluid-carrying catheter for surgical operations such as atherosclerosis peeling.

### BACKGROUND

For the operations such as atherosclerosis peeling, traumatic large blood vessel repairing, thoracic aorta, abdominal aorta, and carotid artery dissection, aneurysm and intracranial artery clipping, many large vessels at present cannot be treated by surgical operations mainly because the important organs cannot be long-term ischemia and hypoxia, and the large blood vessel incision and repair and the excessive bleeding of atherosclerosis will cause the patient's life to be dangerous and death. For example, the current atherosclerosis peeling operation is performed by clamping the arteries at both ends of the lesion with medical clamps, blocking blood from passing through the site of the lesion and then peeling the atherosclerosis and suturing the blood vessels. For the above surgical procedure, if the time is too long, it is prone to a series of complications caused by ischemia and hypoxia of important organs during the operation, and the above-mentioned surgical operations have a large amount of surgical bleeding and require a large amount of plasma.

### SUMMARY OF THE INVENTION

In view of the drawbacks of the prior art, an objective of the present invention is to provide a double balloon fluid-carrying catheter, which is used for operations such as dissection of atherosclerosis, aortic dissection, aortic aneurysm repair, intracranial aneurysm and arterial dissection, coronary artery stenosis, and reduces surgical bleeding and avoids a series of complications caused by ischemia and hypoxia of important organs during surgery, thereby improving the success rate of surgery.

The present invention is implemented by the following technical solutions: A double balloon fluid-carrying catheter, including an outer conduit and a balloon, two luer ports being provided at a proximal end of the outer conduit, one port being used to inflate and deflate the balloon and the other port being used to flush a guidewire lumen, the balloon being provided near a distal end of the outer conduit, wherein the balloon is provided with two balloons, namely a proximal balloon and a distal balloon, and there is a certain distance between the proximal balloon and the distal balloon; a proximal end of the proximal balloon is provided with a plurality of side holes on a wall of the outer conduit, the side holes conduct an inside and an outside of the outer conduit, and a distal end of the distal balloon is provided with a bridging fluid-carrying hole.

For the above double balloon fluid-carrying catheter, on the basis of using the peripheral balloon expansion catheter in prior art (at present, the peripheral balloon expansion catheter is suitable for dilating narrow segments of peripheral blood vessels and arteriovenous dialysis fistulas, and the balloon is inflated to a known diameter according to the specific pressure on the product label and compliance table. a radiopaque marker is provided on each end of the balloons, which is used to assist the fluoroscopic observation and positioning of the balloon catheter in the direction of the lesion, and to facilitate passing through the lesion; the expansion catheter has a soft tapered head to assist in pushing the catheter, and a proximal end of the expansion catheter has two Luer ports, of which one is used to connect a filling device to inflate and deflate the balloon and the other is used to flush a guidewire lumen), the structure with one balloon is modified into a structure with double balloons with a proximal balloon and a distal balloon, a bridging section is formed between the double balloons, and a plurality of side holes are opened on a wall of the proximal outer conduit of the proximal balloon while arranging the distal end of the distal balloon with a bridging fluid-carrying hole. In this way, when atherosclerotic dissection or other surgical operations are performed, the balloon of the double balloon fluid-carrying catheter is first guided to the lesion site through the guide wire so that the proximal balloon and the distal balloon are placed on both sides of the lesion, and then the proximal balloon and the distal balloon are inflated to a known diameter so that the outer surface of the balloon is in close contact with the inner wall of the artery and blood vessel to block blood from passing through the lesion site, and then atherosclerotic dissection or other surgical operations may be performed. During the operation, the blood in the artery will enter the inside of the expansion catheter through the side hole, and then flow out through the bridging fluid-carrying hole, without blocking the blood supply to important organs; therefore, this may not only reduce the amount of surgical bleeding, but more importantly, this may avoid a series of complications caused by ischemia and hypoxia of important organs during the operation.

Preferably, the distal end of the outer conduit is provided with a soft tapered head, and the bridging fluid-carrying hole axially penetrates through the tapered head.

Preferably, both ends of the proximal balloon and the distal balloon are each provided with a radiopaque mark.

Preferably, an outer surface of the outer conduit is provided with a hydrophobic silica gel coating.

### BRIEF DESCRIPTION OF THE DRAWINGS

The present invention has the following drawings:

FIG. 1 is a schematic structural diagram of the present invention;

FIG. 2 is a diagram of the working principle of the present invention in a surgical operation.

### DETAILED DESCRIPTION OF THE EMBODIMENTS

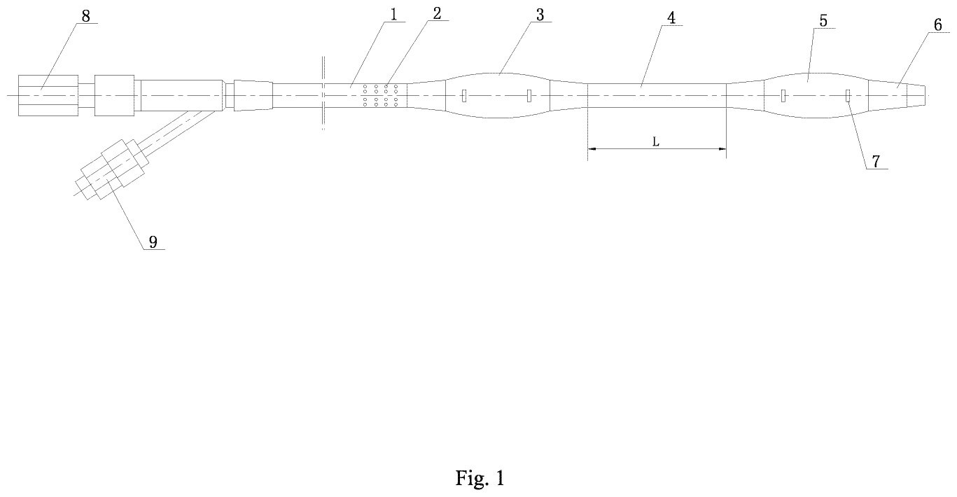

As shown in FIG. 1, the double balloon fluid-carrying catheter, with a length specification of 110 cm or 150 cm, includes an outer conduit 1 and a balloon; an outer surface of the outer conduit 1 is provided with a hydrophobic silica gel coating, and a diameter of the outer conduit 1 may be 4 F, 5 F, 6 F, 7 F, 8 F and other specifications; a proximal end of the outer conduit 1 has two Luer ports, of which one 9 is an inflation port used to inflate and deflate the balloon and the other 8 is used to flush a guidewire lumen; the balloon is provided near a distal end of the outer conduit 1, and the balloon is provided with two balloons, namely a proximal balloon 3 and a distal balloon 5; a radiopaque marker 7 is provided on each end of the proximal balloon 3 and the distal balloon 5 for assisting the positioning of the balloon fluid-carrying catheter during use; the proximal balloon 3 and the distal balloon 5 are separated by a certain distance from each other as a bridging section 4, and a distance L of the bridging section 4 may be selected from 5 cm, 8 cm, 12 cm, 15 cm and other length specifications to adapt to different atherosclerotic vessel lengths; a proximal end of the proximal balloon 3 is provided with a plurality of side holes 2 on a wall of the outer conduit 1, and the side hole 2 connects an inside and an outside of the outer conduit 1; a distal end of the distal balloon 5 is provided with a bridging fluid-carrying hole, the distal end of the outer conduit 1 is provided with a soft tapered head 6, and the bridging fluid-carrying hole axially penetrates through the tapered head 6.

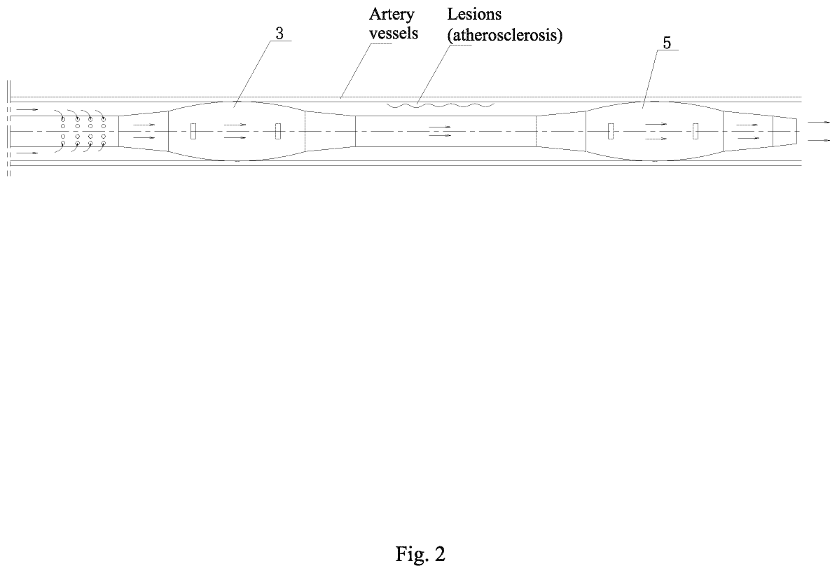

As shown in FIG. 2, the present invention is applied in surgical operations as follows: when atherosclerotic dissection is performed, the balloon of the double balloon fluid-carrying catheter is first guided to the lesion site through the guide wire so that the proximal balloon and the distal balloon are placed on both sides of the lesion, and then the proximal balloon and the distal balloon are inflated to a known diameter so that the outer surface of the balloon is in close contact with the inner wall of the artery and blood vessel to block blood from passing through the lesion site, and then atherosclerotic dissection may be performed. During the operation, the blood in the artery will enter the inside of the expansion catheter through the side hole, and then flow out through the bridging fluid-carrying hole (the blood flow is shown by the arrow in FIG. 2), without blocking the blood supply to important organs; therefore, this may not only reduce the amount of surgical bleeding, but more importantly, this may avoid a series of complications caused by ischemia and hypoxia of important organs during the operation.

1. A double balloon fluid-carrying catheter. comprising an outer conduit and a balloon, two Luer ports being provided at a proximal end of the outer conduit, one port being used to inflate and deflate the balloon and the other port being used to flush a guidewire lumen, the balloon being provided near a distal end of the outer conduit, wherein the balloon is provided with two balloons, namely a proximal balloon and a distal balloon, and there is a certain distance between the proximal balloon and the distal balloon; a proximal end of the proximal balloon is provided with a plurality of side holes on a wall of the outer conduit, the side holes conduct an inside and an outside of the outer conduit, and a distal end of the distal balloon is provided with a bridging fluid-carrying hole.

2. The double balloon fluid-carrying catheter according to claim 1, wherein the distal end of the outer conduit is provided with a soft tapered head, and the bridging fluid-carrying hole axially penetrates through the tapered head.

3. The double balloon fluid-carrying catheter according to claim 1, wherein both ends of the proximal balloon and the distal balloon are each provided with a radiopaque mark.

4. The double balloon fluid-carrying catheter according to claim 1, wherein an outer surface of the outer conduit is provided with a hydrophobic silica gel coating.
